(19) United States
(12) Patent Application Publication
(10) Pub. No.: US 2018/0092571 A1
(43) Pub. Date: Apr. 5, 2018

(54) SYSTEM FOR IN-HOME SELF-ASSESSMENT OF RISK FOR ABNORMAL AIRFLOW DURING SLEEP

(71) Applicant: SleepBit, LLC, Tulsa, OK (US)

(72) Inventors: Steven M. Wood, Tulsa, OK (US); Paul Wylie, Sherwood, AR (US); Bill Ardrey, Cleveland, OK (US); Alex Myers, Tulsa, OK (US)

(73) Assignee: SleepBit, LLC, Tulsa, OK (US)

(21) Appl. No.: 15/723,999

(22) Filed: Oct. 3, 2017

Related U.S. Application Data
(60) Provisional application No. 62/403,977, filed on Oct. 4, 2016.

(51) Int. Cl. A61B 5/087; A61B 5/00; G06F 19/00; A61B 5/11

(52) U.S. Cl. CPC A61B 5/087; A61B 5/4809; A61B 5/682; A61B 5/486; A61B 5/1116; G06F 19/3406

(57) ABSTRACT

A self-assessment system for assessing risk for experiencing abnormal airflow during sleep. A preferred embodiment of the system includes a cannula for sampling nasal airflow of a user. A data recording and communication device includes a sensor to receive airflow from the cannula, a converter to convert breathing airflow to an electronic signal, a recorder to record electronic signal data, and a transmitter to wirelessly transmit said electronic signal data. A mobile device includes a receiver to receive said electronic signal data transmitted from the recording and communication device, a database to store the electronic signal data, an interface to obtain questionnaire data from the user, a transmitter to wirelessly transmit the electronic signal and questionnaire data, and a mechanism to store or access instructional video information. A computer server includes a receiver to receive the electronic signal data and questionnaire data transmitted from the mobile device and a mechanism to generate a risk profile from the data received.

### CROSS REFERENCE TO RELATED APPLICATIONS

This application claims priority to and the benefit of U.S. Provisional Patent Application Ser. No. 62/403,977, filed Oct. 4, 2016, which is incorporated herein in its entirety by reference.

### BACKGROUND OF THE INVENTION

### 1. Field of the Invention

The present invention relates to a self-assessment system for risk of experiencing episodes of abnormal airflow during sleep. In particular, the present invention is directed to a self-assessment system that captures data regarding airflow of a user during wakefulness or sleep and supplements the data with answers to health and lifestyle questions in order to analyze the combined information to produce a risk assessment for experiencing episodes of abnormal airflow during sleep.

### 2. Description of the Related Art

There are, according to the American Academy of Sleep Medicine, between 25 million and 35 million Americans with undiagnosed Obstructive Sleep Apnea (OSA), and a variety of over 50 different sleep disorders. Fifty-five million Americans suffer from insomnia at any one time. The medical community and the public in general are becoming more aware of sleep disorders' serious co-morbidities, such as metabolic problems, hypertension, stroke, mood disorders, and poor quality of life. A significant percentage of the public, however, has been reluctant to enter into screening due to cost and an intimidating and time consuming clinical process. There is no currently available non-prescription in-home self-assessment system for determining risk of experiencing episodes of abnormal airflow during sleep used either alone or in combination with airflow analysis, demographic, and health lifestyle information to produce a computerized analysis.

The present invention is a system which includes a collection of physical devices, software, and algorithms that capture a user's airflow during sleep and compares it to a normal breathing curve. Identification of abnormal airflow during breathing cycles is one of the strongest indicators of risk for possible sleep related breathing disorders. The present invention employs a proprietary cannula connected to a wireless communication enabled data logger/device and attendant smart device software application. The system is integrated and programmed for gathering and transmitting data and receiving commands. The present system, coupled with intuitive functional design, allows ease of use by the non-medically trained. Airflow data is captured by the device and communicated to a server via a specifically developed multi-platform mobile application. The computer software application or app allows the user easy video, voice, and written instruction upon smart device initiation. The system is capable of communicating user data captured, as well as providing real time risk assessment results. Proprietary algorithms analyze captured data, develop comparative curves, and transmit results to the user in an easy to understand format. To augment the physical data, a series of lifestyle or health questions for users has been developed based on years of certified sleep disorder specialists' research and practice. The combined analysis of the survey and airflow yields a risk assessment with a very high positive predictive value and minimal false negatives. This high degree of sensitivity and specificity is accomplished through the proprietary cannula and recorder device design and function and through the use of three unique proprietary algorithms that reflect over two decades of physician accumulated clinical and research-based knowledge of sleep medicine. One algorithm analyzes the raw airflow data, one analyzes and values user demographic and health and lifestyle responses, and one analyzes the outcome from the data and response analysis to create a further weighted and valued summary assessment of risk for the user experiencing abnormal airflow during sleep. The algorithms can be adjusted over time to reflect advances in system design, technology, and clinical and research based medical knowledge.

It is a principal objective of the present invention to provide a home assessment suite of components that, when used as a system, reliably captures airflow, augments the airflow data with user responses to a research and practice based lifestyle questionnaire, communicates the submitted information to a processor, analyzes the data with proprietary algorithms, and then displays risk assessments for personal user health decisions. The system can be remotely reprogrammed for advances or changes in device functionality without having to return the device for updates.

It is a further objective of the present invention to provide a system of home self-assessment which does not replace professional medical assessment and does not make a medical diagnosis, but acts as a preliminary screening mechanism for risk.

These and other objectives will be readily apparent upon reference to the drawings, the specifications, and claims submitted herewith.

### SUMMARY OF THE INVENTION

The present invention is directed to a system for in-home self-assessment for risk of experiencing abnormal airflow during sleep.

The system includes a cannula for gathering airflow data from a user. The cannula has a main tube and an inside diameter specifically defined to deliver an air pressure range parameter.

In one preferred embodiment, the cannula is in turn connected to a data recording and communication device, which includes an internal circuit board with a microprocessor, a short distance wireless communication module, a data memory, capacitors and resistors, and an electromechanical sensor. An electrical analog to digital converter converts airflow pressure received to electronic signal data. The data recording and communication device records the electronic signals and is thereafter capable of transmitting the signals. A mobile device includes a receiver to receive the electronic signal data, which is transmitted from the recording and communication device. A database in the mobile device stores the electronic signal data. The mobile device also includes an interface in order to obtain demographic and health and lifestyle questionnaire responses from a series of questions which are presented to a user. The mobile device also includes a transmitter to wirelessly transmit the electronic signal data and the questionnaire response data.

In an alternate preferred embodiment, the electronic signal data from the data recording device is transmitted to a personal computer, such as via a wired connection.

In a first embodiment, a computer server receives the electronic signal data and the questionnaire response data transmitted from the mobile device. In a second embodiment, the computer server receives the electronic signal data from a personal computer. The data collected at the computer server is analyzed using proprietary algorithms. The airflow pressure from a user is captured over time and compared against a normal airflow curve. A mechanism is provided to generate a summarized risk profile from the combination of the electronic signal data and the questionnaire response data received.

### BRIEF DESCRIPTION OF THE DRAWINGS

FIG. 1 is a perspective view of a data recording and communication device which is a component of an in-home self-assessment system for risk of experiencing episodes of abnormal airflow during sleep of the present invention;

FIG. 2 is a schematic diagram of the components of the data recording and communication device;

FIG. 3 is a perspective view of a cannula which is a component of the self-assessment system for risk of experiencing episodes of abnormal airflow during sleep;

FIG. 4 is a sequential flow chart showing initial set-up of the self-assessment system for risk of experiencing episodes of abnormal airflow during sleep;

FIG. 5 is a schematic diagram of various components of a first preferred embodiment of the self-assessment system for risk of experiencing episodes of abnormal airflow during sleep;

FIG. 6 is a schematic diagram of various components of a second preferred embodiment;

FIG. 7 shows the various options presented to a user from a smart mobile device of the self-assessment system for risk of experiencing episodes of abnormal airflow during sleep; and

FIG. 8 is a sequential flow chart illustrating use of the self-assessment system for risk of experiencing episodes of abnormal airflow during sleep.

### DETAILED DESCRIPTION OF THE INVENTION

The embodiments discussed herein are merely illustrative of specific manners in which to make and use the invention and are not to be interpreted as limiting the scope.

While the invention has been described with a certain degree of particularity, it is to be noted that many modifications may be made in the details of the invention's construction and the arrangement of its components without departing from the scope of this disclosure. It is understood that the invention is not limited to the embodiments set forth herein for purposes of exemplification.

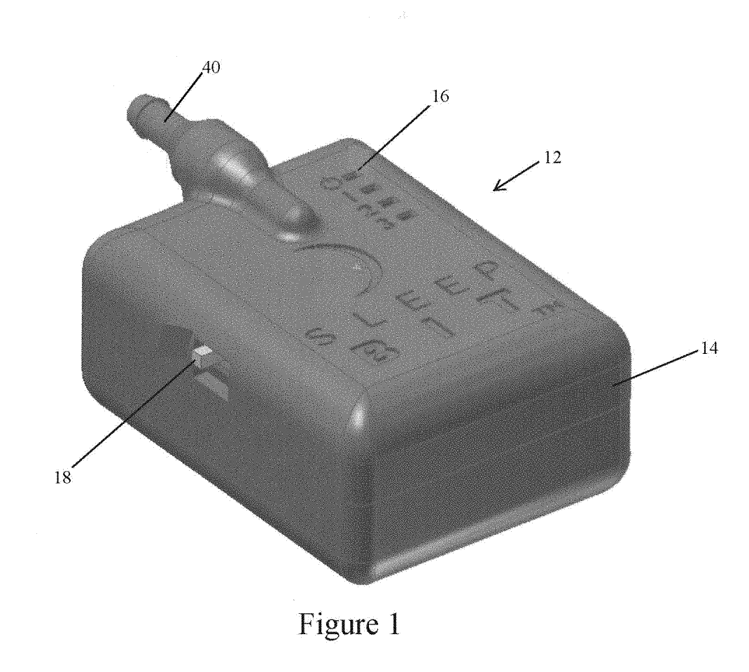

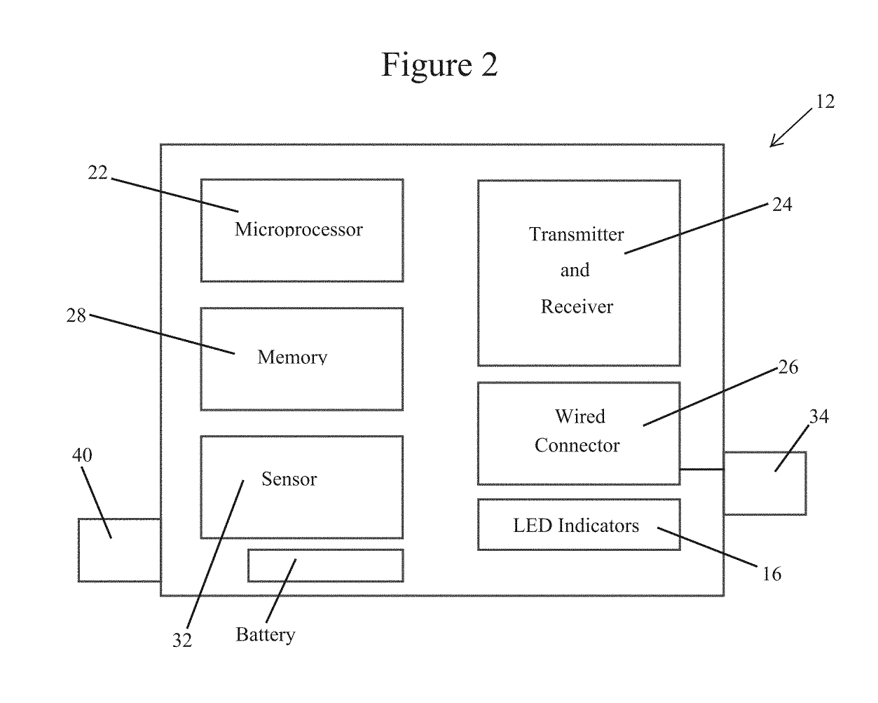

Referring to the drawings in detail, FIG. 1 is a perspective view of a data recording and communication device 12 which is a component of the system 10. The device 12 is comprised of an internal circuit board with a microprocessor 22, a short distance wireless transmitter and receiver module 24 such as Bluetooth™, a connection 26 for wired transmission and receipt of data, a memory 28, capacitors and resistors, as well as a register or sensor 32. FIG. 2 illustrates a schematic diagram of the device 12 showing the various components. The wired connector 26 may include a port, such as a USB connection 34.

In a preferred embodiment, an electromechanical sensor is utilized. The electromechanical sensor or register receives breathing airflow from a cannula and an electrical analog to digital converter converts breathing airflow pressure to an electronic data signal.

In an alternate embodiment, a thermal sensor senses temperature during inhale and exhale breathing cycles and converts it to an electronic data signal.

These components, along with software computer programs contained therein, have the capability to 1) record multiple nights of airflow data during sleep, 2) identify a sufficient quality airflow recording session to warrant communicating the data via Bluetooth™ to a smart mobile device 20, 3) indicate outcomes to the user, and 4) initiate pairing communication with a smart device or, alternatively, with a personal computer for further transmission of the electronic signal data to a computer server 30.

The data recording and communications device 12 has sufficient capacity and memory to record breathing airflow data for multiple nights. A sufficient quality airflow recording session is determined by a number of cycles of breathing data over a period of time with removal of inadequate or bad data received via proprietary algorithms.

As will be described in detail, the electronic signal data is transmitted, analyzed, and reported, which may be communicated with a mobile smart device.

All proprietary calculations occur on the computer server 30. Upon completion of a server calculation routine, the server 30 sends risk profile information to the smart device 20 for display to the user. The server 30 uses proprietary algorithms to process the data and produce a combined assessment profile.

The internal circuit board with memory 28 on the recording and communication device 12 can be reprogrammed remotely by a system administrator to update any aspect of the device displays, communications, and commands.

As shown in FIGS. 1 and 2, the device 12 includes a housing 14 having LED indicators 16, which advise the user about power readiness, data status, and other user/system information. The external features provide functionality which are easily understandable to the user. Instructions and interpretations of the lighting sequence are also defined and displayed on the smart device screen.

Upon attaining sufficient data to analyze, the recording and communication device 12 indicates via LED lighting that sufficient quality data has been recorded for analysis. Various color lighting, blinking sequences, lighting groups, and lighting group blinking sequences from the LED indicators 16 signals the user of various results or actions required. The possible combinations are numerous; however, user instructions advise the user of the specific lighting settings. This flexible functionality coupled with specific user visual indicators results in ease of use for a first-time novice user.

A power button 18 activates the recording and communication device 12 and initiates linking of the recorder device with a smart device 20 or, alternatively, with a personal computer 36. The linking activity also qualifies the user, who must provide a use authorization code identifying the individual who acquired the data logger. The system 10 will not function without the user authorization.

The recording device 12 and mobile smart device 20 are paired via a serialized code which must be matched with an authorized recording device purchase number for the security benefit of the user and for maintaining integrity of data of the authorized user. The pairing specificity and the retention of all user information and data on a protected server ensure, enhance, maintain, and protect user data and assessment specificity and privacy. Such pairing thereby allows communication and data transfer between the recording device 12 and mobile smart device 20.

The device housing 14 also has an integrated receiver 40 for a cannula. The integrated receiver 40 is of specific shape and dimension such that it will receive the cannula so that a positive seal is created, which does not allow air from breathing airflow to escape or air from outside the cannula to invade. No external clamps or other collar reinforcements are required.

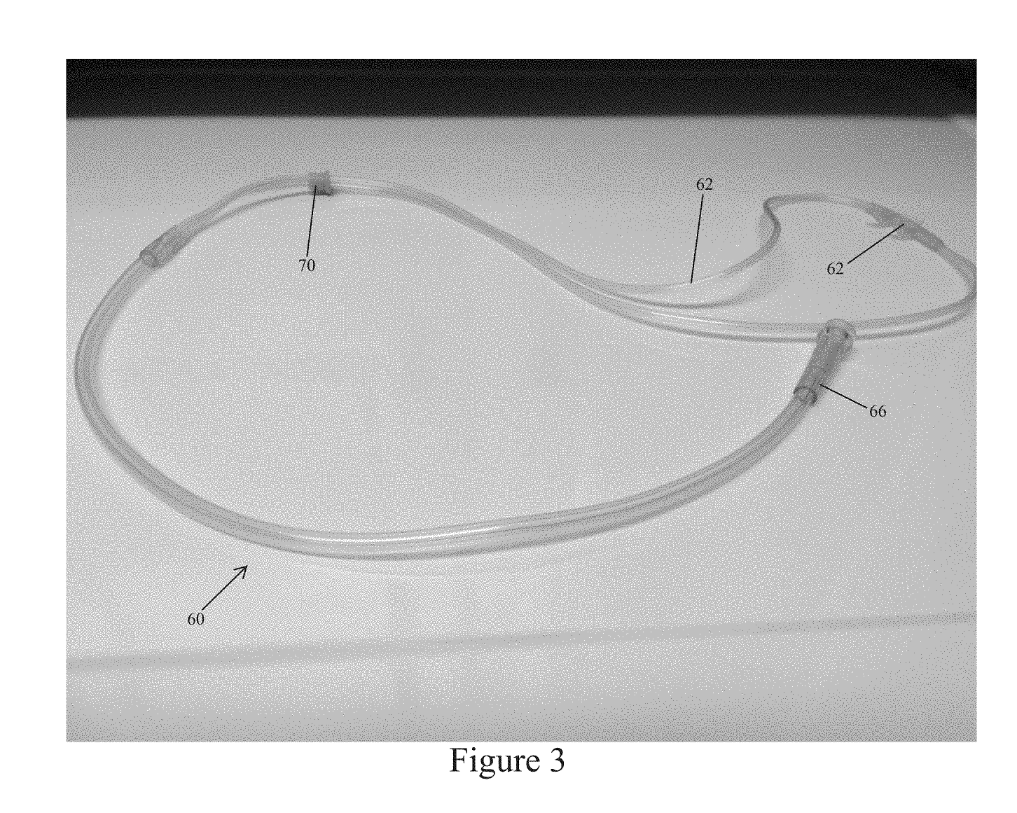

FIG. 3 is a perspective view of the cannula 60, which has a proprietary design both in length and cross-section, creating a unique and specific airflow registry to the data recording device. The cannula 60 connects and remains connected to the recording device 12 by mechanical means and friction with a main connector 66. The cannula 60 fits to the user by placing the head-loop 62 around the user's ears with nose prongs or nostril pieces 64 positioned at the user's nostrils. An optional clip, such as an alligator clip, connects the cannula to night clothes of a user, in order to enhance data quality.

The length of a main tube 68 of the cannula is adjustable by an adjustment mechanism 70. The main tube 68 has a selected length and diameter to deliver an air pressure range. Specific length of the main tube of the cannula 60 allows easy use of the connected recording device without entangling the user or separating from the recording device and allows for improved accuracy of data collection when used in coordination with the system. The cannula is encircled by a plastic band attached to a small alligator clip. Use of the clip to attach the cannula/recorder unit to an article of clothing worn over the chest helps to enhance airflow data recording quality.

The length and internal diameter of the cannula tubing are optimized to allow sufficient pressure at the sensor to pick up airflow without interference from outside factors. Stated in other words, the cannula dimensions are coordinated with cycles of breathing and pause.

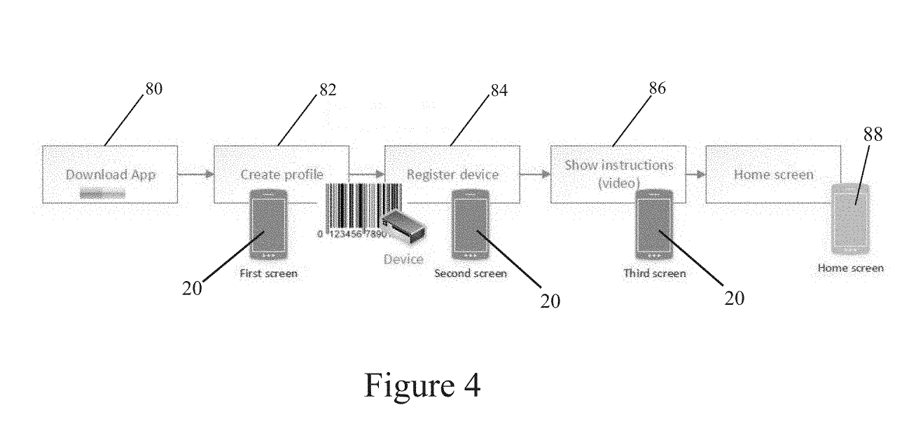

FIGS. 4, 5, 6, and 7 represent the system 10 and the components and application configuration which allow a user to connect with the system 10, receive instructions, communicate data, and receive results.

FIG. 4 is a flow chart showing the initial set-up of the system 10. A software application is downloaded on a mobile smart device 42, such as a cell phone, as seen at box 80. Thereafter, the user is prompted by a series of screens to interact with the device to create a user profile, as seen at box 82. The user will also be prompted to enter an order number, which will be coordinated with the user profile. This may be communicated by entry of an alphanumeric code or by scanning. These actions will create an account for the particular user.

As shown at box 84, the system will then be registered. A video or videos or links to a video or videos will be presented to the user, as shown at box 86. Finally, a home screen will be presented to the user, as shown at box 88.

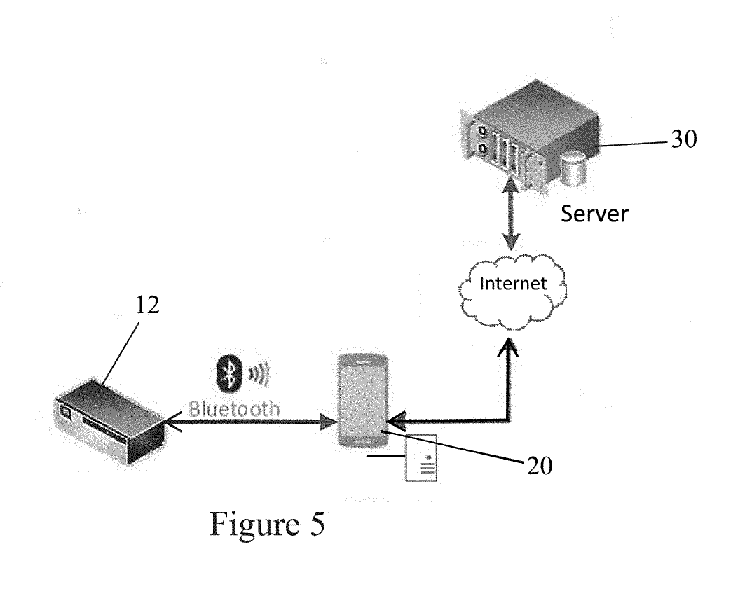

FIG. 5 illustrates the data flow for a first preferred embodiment illustrating a fully wireless configuration of the present invention.

The recording device 12, the smart device 20, and the server 30 may be wirelessly connected. Transmission of data, instructions and results occurs between devices. Data is gathered from the user through the cannula 60 by the recording device 12 and has sufficient power and memory to record more than one typical sleep session. The recording device 12 measures the magnitude and frequency of breaths of an individual. Abnormal events are scored, such as a decline in magnitude compared to earlier breathing.

It is not a requirement that the mobile smart device 20 be powered during data gathering sleep sessions. Moreover, the mobile smart device 20 does not have to be under the user's pillow or even in the sleeper's vicinity. This is a unique feature of the system 10, which results in improved functionality for users, avoids power drains on their smart device, and avoids data and usage charges by their data service or telephone service provider.

The mobile smart device 12 is the primary point of communication with the user. As shown on FIG. 4, the user must initially download a mobile device software application (app) from which a User Profile is created. Thereafter, registry of the recording device is accomplished, as shown at box 84. Instructional information (written, pictorial, video, and/or verbal) is provided to the user, as shown at box 86.

As seen in a first preferred embodiment in FIG. 5, upon data collection of sufficient quality, quantity and duration, data transfer occurs when the user pairs the recording device 12 with the mobile smart device 20. This data is further communicated to the server 30, such as through the internet. Analysis of the data collected is performed at the computer server 30 using proprietary algorithms. No calculation capacity need reside on the recording device 12 or mobile smart device 20. The use of the computer server 30 provides unique protection of user demographic and health information beyond expected standards for a mobile device. Such features are both for security of user data and for reduction of processing capacity on the recording and communication device 12 and on the mobile smart device 20.

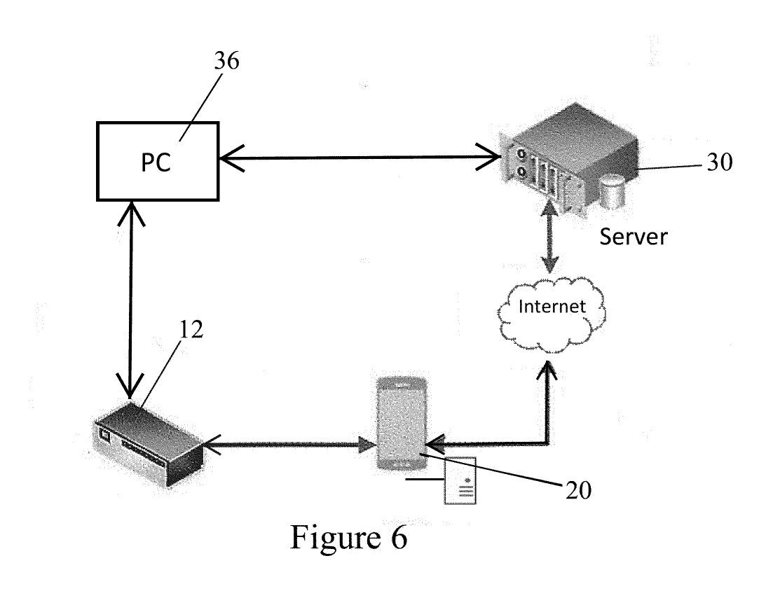

FIG. 6 illustrates a second preferred embodiment of the invention utilizing a wired connection from the recording device 12 to a personal computer 36. Upon data collection of sufficient quality, quantity, and duration, data transfer occurs between the recording device 12 and a central processing unit, such as a desktop or laptop computer 36. The data is further communicated to a computer server 30, such as through the internet. Analysis of the data is performed at the computer server using proprietary algorithms. No calculation capacity need reside on the recording device 12 nor on the central processing unit 36.

A series of life and health questions are posed to the user by the mobile device. Answers to the lifestyle and health questionnaire are transmitted between the mobile smart device 20 and the computer server 30.

A user risk profile generated is calculated using the combined input of both the airflow data results and the questionnaire results. Calculations by the proprietary algorithms determine the final summarized risk profile.

A series of demographic, health, and lifestyle questions are posed to the user by the mobile device 20. By way of example but not by way of limitation, questions posed to users may include whether a user is aware of snoring and to what extent and duration, whether a user feels sleepiness at various times, whether a user has experienced physical symptoms during sleep, whether a user has used tobacco or caffeine, whether a user takes certain medications, and various other history and lifestyle questions. It is possible to present information to a user in response to answers provided. For example, incentive educational facts may be presented to enhance the user experience.

Responses to the lifestyle and health questionnaire are transmitted between the mobile smart device 20 (where such questions are answered) and the computer server 30. The questionnaire may be in two different forms: an abbreviated short form version and a more comprehensive longer form version. Two uniquely different health and lifestyle questionnaires have been developed: an abbreviated module using just a few questions that allows those users so inclined to move very rapidly through their assessment process, and a second highly comprehensive module that allows those users so inclined to provide more extensive health and lifestyle information that further enhances the accuracy of their risk assessment. Included in the more comprehensive module as a purposeful unique design feature are continually updatable educational sleep related facts that serve to enhance user value and experience and to incentivize and reward completion of the comprehensive assessment process.

The user risk profile is calculated using both the airflow data results and the questionnaire results. Calculations by the proprietary algorithms determine the final summarized risk profile.

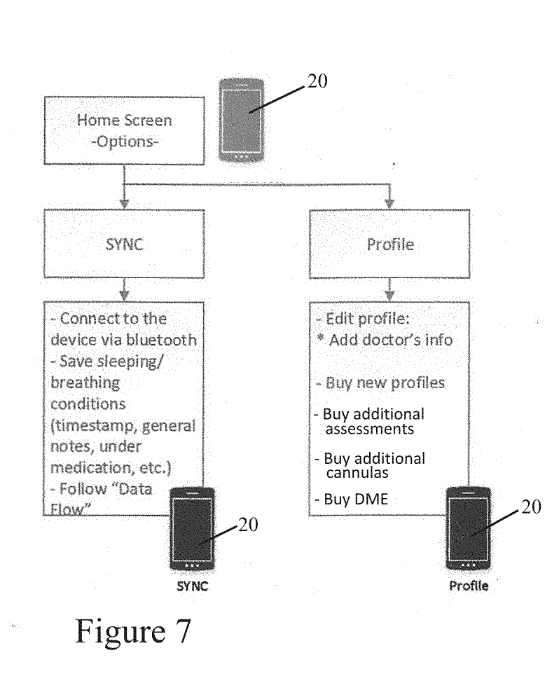

The mobile device 20 may have home screens presented to the user that have various options, as shown in FIG. 7. The functional aspects of the data and information flows are a series of user cycles, periodically synchronized with updated profiles.

For example, under the designation “Sync”, the various sleeping and breathing data may be saved. Additionally, under the designation “Profile”, there is an ability to edit the user profile and add further information.

It will be appreciated that the configuration shown is by way of demonstration and does not represent the full array of possible options that can be performed.

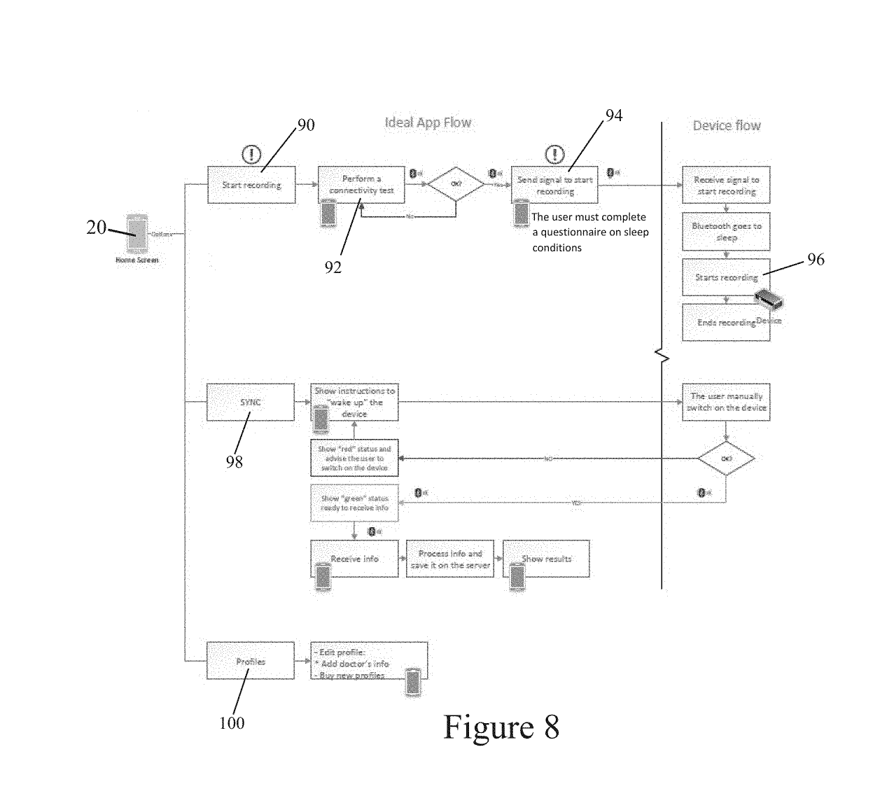

Finally, FIG. 8 illustrates a sequential flow chart illustrating use of the present invention. Initially, a command can be entered from the mobile device 20 to start recording a sleep cycle, as shown at box 90. Thereafter, a connectivity test is performed, as shown at box 92. If connection is made, a signal is sent from the mobile device to the recording device 12 to begin, as shown at box 94. Thereafter the recording device 12 begins to record breathing data received through the cannula from the user, as shown at box 96.

The system 10 also has a process to sync or synchronize data, as shown at box 98. Data gathered from a breathing session is transmitted to the mobile device 20 and thereafter to the computer server 30.

The system also has a process to edit a user profile, add profiles, or edit information, as shown at box 100.

Whereas, the invention has been described in relation to the drawings attached hereto, it should be understood that other and further modifications, apart from those shown or suggested herein, may be made within the scope of this invention.

What is claimed is:

1. A breathing self-assessment system for risk of experiencing abnormal airflow during sleep, which system comprises:
a cannula for sampling nasal breathing airflow pressure of a user;
a data recording and communication device including
a. a sensor to receive breathing airflow from said cannula,
b. a converter to convert breathing airflow to electronic signal data,
c. a recorder to record said electronic signal data, and
d. a transmitter to transmit said electronic signal data;
a mobile device including
a. a receiver to receive said electronic signal data from said recording and communication device,
b. a database to store said electronic signal data,
c. an interface to obtain health and lifestyle questionnaire response data from a user,
d. a transmitter to wirelessly transmit said electronic signal data and said questionnaire response data, and
e. a mechanism to store or access instructional video information;
a computer server including
a. a receiver to receive said electronic signal data;
b. a receiver to receive said questionnaire response data transmitted from said mobile device, and
c. a mechanism to analyze and generate a risk profile from a combination of said electronic signal data and said questionnaire response data received.

2. A breathing self-assessment system as set forth in claim 1 wherein said data recording device receives and transmits wireless communication to and from said mobile device and wherein said mobile device receives and transmits wireless communication to and from said data recording device.

3. A breathing self-assessment system as set forth in claim 2 wherein said wireless communication is via Bluetooth.

4. A breathing self-assessment system as set forth in claim 1 including a personal computer in communication with said data recording and communication device via a wired connector and wherein said personal computer receives said electronic signal data from said recording and communication device.

5. The breathing self-assessment system as set forth in claim 1 wherein said sensor is an electromechanical sensor and wherein said converter is an electrical analog to digital converter.

6. The breathing self-assessment system as set forth in claim 1 wherein said sensor is a thermal registry sensor and wherein said converter is a temperature to digital converter.

7. The breathing self-assessment system as set forth in claim 1 wherein said mechanism to generate a risk profile includes capturing said breathing airflow from a user over time and comparing against normal airflow pressure curve models.

8. The breathing self-assessment system as set forth in claim 1 wherein said interface to obtain questionnaire data from a user includes a series of questions on sleep habits and other behaviors of said user.

9. The breathing self-assessment system as set forth in claim 1 wherein said mechanism to analyze and generate a risk profile removes inadequate or bad electronic signal data.

10. A data recording and communication device for a breathing self-assessment system, which device comprises:
a microprocessor having a memory;
a sensor to receive breathing airflow from a cannula;
an electrical analog to digital converter to convert breathing airflow to electronic signal data;
a recorder to record said electronic signal data;
a wireless transmitter and receiver; and
a portable power supply.
